(19) United States
(12) Patent Application Publication
(10) Pub. No.: US 2024/0389885 A1
(43) Pub. Date: Nov. 28, 2024

(54) DAILY FASTING METHANE TO DETECT INTESTINAL METHANOGEN OVERGROWTH AND MONITOR TREATMENT RESPONSE

(71) Applicant: Cedars-Sinai Medical Center, Los Angeles, CA (US)

(72) Inventors: Ali Rezaie, Beverly Hills, CA (US); Mark Pimentel, Los Angeles, CA (US)

(73) Assignee: Cedars-Sinai Medical Center, Los Angeles, CA (US)

(21) Appl. No.: 18/695,144

(22) PCT Filed: Oct. 17, 2022
(86) PCT No.: PCT/US2022/078220

§ 371 Date: Mar. 25, 2024

Related U.S. Application Data
(60) Provisional application No. 63/257,256, filed on Oct. 19, 2021.

(51) Int. Cl. A61B 5/08; A61B 5/00

(52) U.S. Cl. CPC A61B 5/082; A61B 5/4255; A61B 5/4848; G01N 33/4975; G01N 33/4977; G01N 2800/065

(57) ABSTRACT

Described herein are methods of utilizing single fasting methane breath concentrations to monitor, select treatment for, and to treat subjects having intestinal methanogen overgrowth.

### CROSS-REFERENCE TO RELATED APPLICATIONS

This application includes a claim of priority under 35 U.S.C. § 119 (e) to U.S. provisional patent application No. 63/257,256, filed Oct. 19, 2021, the entirety of which is hereby incorporated by reference.

### FIELD OF INVENTION

This invention relates to methods of monitoring intestinal methanogen overgrowth treatment and selection of treatment.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Intestinal methanogens such as Methanobrevibacter smithii produce methane (CH₄) as a byproduct of their metabolism. CH₄ has been shown to slow intestinal transit, and to decrease ileal peristaltic velocity by acting as a gasotransmitter affecting the cholinergic nervous system. Intestinal Methanogen Overgrowth (IMO), where excessive amounts of methanogens reside in the intestines, has been associated with bloating, gas, abdominal discomfort and constipation. Reduction of CH₄ levels in IMO patients correlates with improvement of symptoms. Treatment usually consists of a 10 to 14-day course of antibiotics such as rifaximin and neomycin.

Currently, IMO is diagnosed using a 2-hour breath test where patients are asked to ingest a sugar substrate (e.g. glucose or lactulose) and have breath samples collected every 15 minutes. These samples are analyzed by gas chromatography to measure the concentration of CH₄ where a CH₄≥10 ppm at any timepoint is diagnostic of IMO.

These tests can be cumbersome and inaccessible for some patients. There remains a need for a need in the art to test for IMO as well as monitor IMO that are more accessible and cost efficient for patients.

### SUMMARY OF THE INVENTION

The following embodiments and aspects thereof are described and illustrated in conjunction with compositions and methods which are meant to be exemplary and illustrative, not limiting in scope.

Various embodiments provide for a method of assessing treatment response in a subject, comprising: obtaining a fasting single breath sample from the subject, wherein the subject is undergoing treatment for intestinal methanogen overgrowth (IMO) or have undergone treatment for IMO; measuring the methane concentration in the fasting single breath sample; comparing the methane concentration to an initial breath methane concentration from the subject or a previous breath methane concentration from the subject, detecting a decrease in the methane concentration in the fasting breath sample compared to the initial breath methane concentration, or compared to the previous breath methane concentration, or detecting a stable methane concentration in the fasting breath sample compared to the initial breath methane concentration, or compared the previous breath methane concentration, or detecting an increase in the methane concentration in the fasting breath sample compared to the initial breath methane concentration, or compared to the previous breath methane concentration, or detecting a methane concentration of less than 10 ppm.

In various embodiments, the initial breath methane concentration from the subject can be from a lactulose breath test, from a glucose breath test, or from a single fasting single methane measurement, or the previous breath methane concentration from the subject can be from a lactulose breath test, from a glucose breath test, or from single a fasting single methane measurement.

In various embodiments, the method can further comprise performing all the method steps two or more times. In various embodiments, the method can further comprise performing all the method steps 3, 4, 5, 6, 7, 8, 9, 10, 11, 12, 13, 14, 15, 16, 17, 18, 19, 20, or 21 or more times. In various embodiments, the method can further comprise comprising performing all the method steps two or more times for about 7-14 days. In various embodiments, the method can further comprise comprising performing all the method steps two or more times for about 10 days.

In various embodiments, all the method steps are performed once per day.

In various embodiments, detecting a decrease in the methane concentration in the fasting breath sample compared to the initial breath methane concentration, or compared to the previous breath methane concentration can indicate that the treatment for IMO is effective, or detecting a stable methane concentration in the fasting breath sample compared to the initial breath methane concentration can indicate that the treatment for IMO is ineffective, or detecting a stable methane concentration in the fasting breath sample compared the previous breath methane concentration can indicate that the treatment for IMO is effective, or detecting an increase in the methane concentration in the fasting breath sample compared to the initial breath methane concentration, or compared to the previous breath methane concentration can indicate that the treatment for IMO is ineffective, or detecting a methane concentration of less than 10 ppm can be indicative of the treatment for IMO is effective.

In various embodiments, the method can further comprise having the subject continue treatment for IMO if the treatment is effective. In various embodiments, the method can further comprise having the subject stop the treatment for IMO if the treatment is ineffective. In various embodiments, the method can further comprise having the subject change the treatment for IMO if the treatment is ineffective or there is recurrence of IMO.

In various embodiments, the method can further comprise having the subject stop the treatment for IMO if the methane concentration is less than 10 ppm.

Various embodiments of the invention provide for a method of monitoring breath methane concentration in a subject, comprising: obtaining a single fasting breath sample from the subject, wherein the subject is undergoing treatment for intestinal methanogen overgrowth (IMO) or has undergone treatment for IMO; measuring the methane concentration in the single fasting breath sample; comparing the methane concentration to an initial breath methane concentration from the subject or a previous breath methane concentration from the subject; and detecting a decrease in the methane concentration in the fasting breath sample compared to the initial breath methane concentration, or the previous breath methane concentration, or detecting a stable methane concentration in the fasting breath sample compared to the initial breath methane concentration, or the previous breath methane concentration, or detecting an increase in the methane concentration in the fasting breath sample compared to the initial breath methane concentration, or the previous breath methane concentration.

In various embodiments, the initial breath methane concentration from the subject can be from a lactulose breath test, from a glucose breath test, or from a fasting single methane measurement, or the previous breath methane concentration from the subject can be from a lactulose breath test, from a glucose breath test, or from a fasting single methane measurement.

In various embodiments, the method can further comprise performing all the method steps two or more times. In various embodiments, the method can further comprise performing all the method steps 3, 4, 5, 6, 7, 8, 9, 10, 11, 12, 13, 14, 15, 16, 17, 18, 19, 20, or 21 or more times. In various embodiments, the method can further comprise performing all the method steps two or more times for about 7-14 days. In various embodiments, the method can further comprise performing all the method steps two or more times for about 10 days.

In various embodiments, performing all the method steps can comprise performing all the method steps once per day.

Various embodiments of the present invention provide for a method for treating intestinal methanogen overgrowth (IMO) in a subject, comprising: obtaining a fasting single breath methane concentration from a subject who is undergoing treatment for intestinal methanogen overgrowth (IMO); comparing the fasting single breath methane concentration to an initial breath methane concentration from the subject or a previous breath methane concentration from the subject; and continuing the treatment for IMO if a decrease in the single fasting breath methane concentration compared to an initial breath methane concentration, or compared to a previous breath methane concentration is detected indicating that the treatment for IMO is effective, or stopping the treatment for IMO if a stable single fasting breath methane concentration compared to the initial breath methane concentration is detected indicating that the treatment for IMO is ineffective, or continuing the treatment for IMO if a stable single fasting breath methane concentration compared to the previous breath methane concentration is detected indicating that the treatment for IMO is effective, or changing the treatment for IMO if an increase in the single fasting breath methane concentration compared to the initial breath methane concentration, or compared to the previous breath methane concentration is detected indicating that the treatment for IMO is ineffective, or stopping the treatment for IMO if a methane concentration of less than 10 ppm is detected.

Various embodiments of the invention provide for a method of treating intestinal methanogen overgrowth (IMO) in a subject undergoing treatment for IMO, comprising: continuing the treatment for IMO if a decrease in a single fasting breath methane concentration compared to an initial breath methane concentration, or compared to a previous breath methane concentration is detected indicating that the treatment for IMO is effective, or stopping the treatment for IMO if a stable single fasting breath methane concentration compared to the initial breath methane concentration is detected indicating that the treatment for IMO is ineffective, or continuing the treatment for IMO if a stable single fasting breath methane concentration compared to the previous breath methane concentration is detected indicating that the treatment for IMO is effective, or changing the treatment for IMO if an increase in the single fasting breath methane concentration compared to the initial breath methane concentration, or compared to the previous breath methane concentration is detected indicating that the treatment for IMO is ineffective, or stopping the treatment for IMO if a methane concentration of less than 10 ppm is detected.

Various embodiments of the present invention provide for a method of confirming eradication or lack of eradication of intestinal methanogen overgrowth (IMO), comprising: obtaining a single fasting breath sample from the subject, measuring the methane concentration in the single fasting breath sample; and detecting a methane concentration in the fasting breath sample of less than 10 ppm to confirm eradication of IMO, or detecting a methane concentration in the fasting breath sample of >10 ppm to confirm the lack of eradication of IMO.

In various embodiments, the subject is undergoing treatment for IMO. In various embodiments, subject has undergone treatment for IMO.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts EASE-DO Clinical Study Design for subjects in the placebo arm in accordance with various embodiments of the present invention. Baseline weekly CSBM and SBM were recorded for two weeks prior to initiation of placebo. Subjects received placebo from day 1 to day 84 (12-week period) and continued to record their CSBM and SBM. CH₄ measurements were taken every 4 weeks. CH₄=methane, CSBM=complete spontaneous bowel movement, SBM=spontaneous bowel movement.

FIG. 2 depicts the difference in symptom severity for those with SMM ≥10 ppm vs those with SMM <10 ppm graded on a scale of 0-9, in accordance with various embodiments of the present invention. There was a significant difference in severity for constipation (5.65±3.47 vs 4.32±3.62, p=0.008), gas (6.27±2.77 vs 5.41±2.98, p=0.003) and diarrhea (3.68±3.49 vs 4.38±3.46, p=0.04) while bloating was numerically higher for SMM ≥10 ppm (6.24±3.29 vs 5.71±3.36, p=0.059).

FIG. 3 shows that SMM cutoff of 10 ppm provided the largest difference in constipation score (graded from 0-9) in accordance with various embodiments of the present invention. There was no significant difference for cutoff values ≤7 ppm. SMM=single CH₄ measurement.

FIG. 4 depicts SMM taken during the EASE-DO trial in accordance with various embodiments of the present invention. For subjects participating in EASE-DO trial who received placebo, SMM did not change over time (n=20). SMM=single CH₄ measurement.

FIG. 5 shows that fecal Methanobrevibacter smithii load is positively associated with SMM in accordance with various embodiments of the present invention. SMM=single CH₄ measurement.

FIG. 6 shows that daily SMM levels rapidly and significantly drop during antibiotic therapy. SMM=single CH₄ measurement in accordance with various embodiments of the present invention.

FIG. 7 shows weekly CSBM for subjects receiving placebo in EASE-DO trial in accordance with various embodiments of the present invention. For subjects participating in EASE-DO trial who received placebo, CSBM increased over time. Week 0 represents CSBM recorded during the first 2 weeks prior to receiving placebo.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

As used herein the term “fasting breath sample” refers to a breath sample obtained from a subject who has not consumed food for at least 4 hours.

Unlike hydrogen-predominant SIBO where H₂ decreases significantly during fasting and rises steadily over time as bacteria ferment sugar substrates, IMO patients have been observed to have elevated fasting CH₄ levels with less incremental changes in breath CH₄ after ingesting sugar. As such, a 2-hour breath test may not be needed for the diagnosis of IMO, which could significantly improve patient access and costs. Moreover, a single CH₄ level can serve as a practical biomarker for monitoring treatment success/failure and disease recurrence.

Using two large independent breath test databases, we determined the diagnostic accuracy and clinically meaningful cut-off of a fasting single CH₄ measurement (SMM) to diagnose IMO as compared to a 2-hour lactulose or glucose breath test. Secondly, we assessed the symptoms associated with elevated SMM. Thirdly, we assessed the temporal stability and accuracy of SMM as part of a prospective clinical trial. Fourthly, we investigated the correlation of SMM levels with fecal loads for M. smithii, the predominant archaeon responsible for CH₄ production in humans. Lastly, we conducted a pilot study to assess the utility of daily SMM as an on-treatment monitoring biomarker in subjects undergoing antibiotic therapy for IMO.

Described herein, a single fasting exhaled CH₄ measurement can accurately diagnose intestinal methanogen overgrowth (IMO). Using the cutoff of CH₄≥10 ppm, single methane measurement (SMM) has a sensitivity of 86.4% and a specificity of 100% on both the lactulose and glucose breath test. Also, SMM appears to be stable over time in subjects who do not receive treatment (p=0.45). Furthermore, SMM correlates with stool M. smithii load (R=0.63 p<0.0001) and SMM≥10 ppm is positively associated with the severity of constipation (5.65±3.47 vs 4.32±3.62, p=0.008). Given that SMM ≥10 ppm shares similar test characteristics between glucose and lactulose breath test and has the largest difference in the severity of constipation, 10 ppm appear to be the most clinically meaningful cut-off for SMM.

We show high sensitivity and specificity for SMM in diagnosing IMO on the lactulose breath test. In this study, we further show that SMM can accurately diagnose IMO based on the glucose breath test, and that SMM measured 2 weeks apart from a 2-hour breath test has a sensitivity of 84.8% (95% CI: 75.6-93.9) when using a cutoff of ≥10 ppm. These data indicate that SMM can diagnose IMO with acceptable accuracy irrespective of sugar substrate. In addition, SMM can accurately diagnose IMO when performed independently of the 2-hour breath test on a separate date.

Subjects with SMM ≥10 ppm reported higher scores on constipation and gas, and a lower score on diarrhea. This finding corroborates the physiological role of CH₄ in slowing intestinal transit. Pimentel et al. (Methane, a gas produced by enteric bacteria, slows intestinal transit and augments small intestinal contractile activity. Am J Physiol Gastrointest Liver Physiol. 2006; 290 (6):G1089-95) showed that infusion of CH₄ slows canine small bowel transit and breath CH₄ has been shown to be associated with decreased small bowel and colonic transit in humans (5, 24). Bloating was another symptom that was numerically worse in subjects with elevated SMM, and which has been shown to improve with antibiotic therapy in subjects with CH₄ and IBS-C. Of note, we saw an increase in the severity of constipation as SMM increased, which is suggestive of a biological gradient. According to the Bradford-hill criteria, such a relationship supports a causal relationship, providing further evidence that CH₄ is causing constipation in a subset of patients with IBS-C.

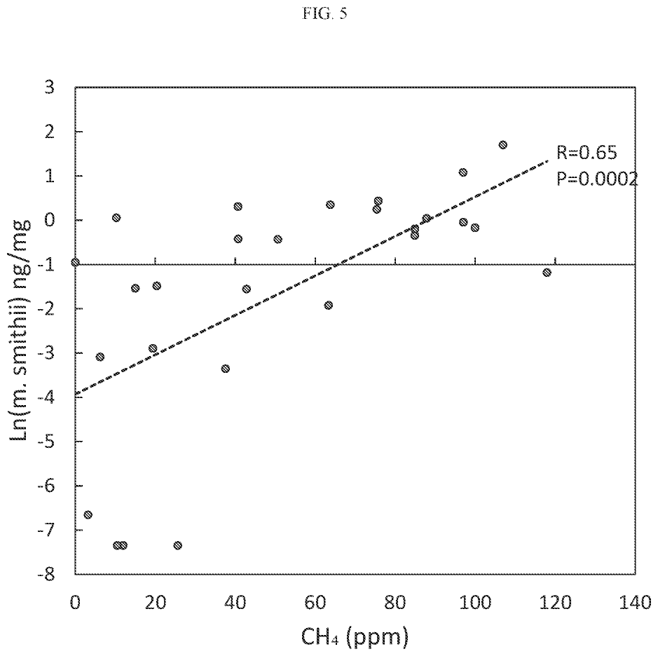

Currently, IMO cannot be diagnosed via duodenal aspiration due to limitation of culturing archaea in clinical microbiology laboratories. In addition, a recent study by Cangemi et al. has shown a modest rate of contamination in duodenal aspirates obtained using standard techniques, making the breath test a more clinically-relevant test for diagnosis of IMO. SMM gives a simple, non-invasive method of performing association studies to measure intestinal CH₄ which may indirectly measure M. smithii loads (see e.g., FIG. 5).

While SMM remained stable when subjects received placebo over 12 weeks, it reliably decreased with antibiotic therapy in IMO patients. SMM can be a simple and inexpensive tool to monitor treatment response in IMO patients. Our data shows that SMM may be useful in confirming eradication or lack of eradication of IMO, which can be useful in tailoring management of IMO. This is akin to the recommendation for confirming H. pylori eradication for patients with dyspepsia.

The strength of our study is the multi-level and comprehensive methodology in assessing SMM as both a diagnostic test and a biomarker for treatment response. Our data were collected form three different cohorts at two different centers to account for discrepancies in location and methodology. Our results showed remarkable similarity across cohorts using the SMM cutoff of 10 ppm for diagnosing IMO. In addition, we show that SMM is associated with constipation and stool M. smithii loads. Furthermore, SMM was stable over time and decreased during antibiotic therapy, which suggests its role as a diagnostic and monitoring biomarker for IMO.

In summary, SMM with a cutoff of 10 ppm accurately diagnoses IMO, is associated with constipation, and positively correlates with stool M. smithii. SMM can be an inexpensive, non-invasive biomarker for intestinal methanogen load.

Accordingly, various embodiments of the present invention are based, at least in part, on these findings.

Various embodiments of the present invention provide for a method of assessing treatment response in a subject, comprising: obtaining a fasting single breath sample from the subject, wherein the subject is undergoing treatment for intestinal methanogen overgrowth (IMO) or has undergone treatment for IMO; measuring the methane concentration in the fasting single breath sample; comparing the methane concentration to an initial breath methane concentration from the subject or a previous breath methane concentration from the subject, detecting a decrease in the methane concentration in the fasting breath sample compared to the initial breath methane concentration, or compared to the previous breath methane concentration, or detecting a stable methane concentration in the fasting breath sample compared to the initial breath methane concentration, or compared the previous breath methane concentration, or detecting an increase in the methane concentration in the fasting breath sample compared to the initial breath methane concentration, or compared to the previous breath methane concentration, or detecting a methane concentration of less than 10 ppm.

Various embodiments of the present invention provide for a method of assessing treatment response in a subject, comprising: obtaining a fasting single breath sample from the subject, wherein the subject is undergoing treatment for intestinal methanogen overgrowth (IMO) or has undergone treatment for IMO; measuring the methane concentration in the fasting single breath sample; comparing the methane concentration to an initial breath methane concentration from the subject or a previous breath methane concentration from the sample; and detecting a decrease in the methane concentration in the fasting breath sample compared to the initial breath methane concentration, or compared to the previous breath methane concentration.

Various embodiments of the present invention provide for a method of assessing treatment response in a subject, comprising: obtaining a fasting single breath sample from the subject, wherein the subject is undergoing treatment for intestinal methanogen overgrowth (IMO) or has undergone treatment for IMO; measuring the methane concentration in the fasting single breath sample; comparing the methane concentration to an initial breath methane concentration from the subject or a previous breath methane concentration from the sample; and detecting a stable methane concentration in the fasting breath sample compared to the initial breath methane concentration, or compared the previous breath methane concentration.

Various embodiments of the present invention provide for a method of assessing treatment response in a subject, comprising: obtaining a fasting single breath sample from the subject, wherein the subject is undergoing treatment for intestinal methanogen overgrowth (IMO) or has undergone treatment for IMO; measuring the methane concentration in the fasting single breath sample; comparing the methane concentration to an initial breath methane concentration from the subject or a previous breath methane concentration from the sample; and detecting an increase in the methane concentration in the fasting breath sample compared to the initial breath methane concentration, or compared to the previous breath methane concentration.

Various embodiments of the present invention provide for a method of assessing treatment response in a subject, comprising: obtaining a fasting single breath sample from the subject, wherein the subject is undergoing treatment for intestinal methanogen overgrowth (IMO) or has undergone treatment for IMO; measuring the methane concentration in the fasting single breath sample; and detecting a methane concentration of less than 10 ppm.

Various embodiments of the present invention provide for a method of assessing treatment response in a subject, comprising: obtaining a fasting single breath sample from the subject, wherein the subject is undergoing treatment for intestinal methanogen overgrowth (IMO) or has undergone treatment for IMO; measuring the methane concentration in the fasting single breath sample; and detecting a methane concentration of greater than 10 ppm.

In various embodiments, the treatment for IMO comprises antibiotic therapy. In various embodiments, the antibiotic therapy comprises a course of rifaximin. In various embodiments, the antibiotic therapy comprises a course of rifaximin and neomycin.

In various embodiments, the treatment for IMO comprises a statin therapy. In various embodiments, the treatment comprises a course of lovastatin.

In various embodiments, the treatment for IMO comprises an elemental diet.

In various embodiments, the initial breath methane concentration from the subject is from a lactulose breath test, from a glucose breath test, or from a single fasting single methane measurement. In various embodiments, the previous breath methane concentration from the subject is from a lactulose breath test, from a glucose breath test, or from single a fasting single methane measurement.

In various embodiments, the method further comprises performing all the method steps two or more times. In various embodiments, the method can further comprise performing all the method steps 3, 4, 5, 6, 7, 8, 9, 10, 11, 12, 13, 14, 15, 16, 17, 18, 19, 20, or 21 or more times. For example, the method is performed once each day while the efficacy of the treatment for the subject is monitored. In other instances, the method is performed after treatment to determine whether the benefit is sustained or whether IMO has returned.

In various embodiments, the method further comprises performing all the method steps two or more times for about 7-14 days. For example, the method steps are performed once each day for about 1-2 weeks. In various embodiments, the method further comprises performing all the method steps two or more times for about 10 days. For example, the method steps are performed once each day for about 10 days. In various embodiments, the method further comprises performing all the method steps two or more times for about 7-10 days. For example, the method steps are performed once each day for about 7-10 days. Monitoring the subject can be done for a shorter period of time (e.g., 3, 4, 5, or 6 days) or a longer period of time (e.g., 15, 16, 17, 18, 19, 20, or 21 days). Monitoring for longer than 21 days can also be done.

In various embodiments, the method further comprises performing all the method steps once per day. That is, one fasting single breath methane test is done each day, and the results are compared to, for example, the initial breath methane level, or a previous breath methane breath level, or several previous breath methane breath levels so as to detect a trend regarding the efficacy of treatment.

In various embodiments, the method further comprises performing all the method steps twice per day. Performing the all the method steps multiple times per day can also be done (e.g., 3 times, 4 times, 5 times, etc.).

In various embodiments, detecting a decrease in the methane concentration in the fasting breath sample compared to the initial breath methane concentration, or compared to the previous breath methane concentration indicates that the treatment for IMO is effective.

In various embodiments, detecting a stable methane concentration in the fasting breath sample compared to the initial breath methane concentration indicates that the treatment for IMO is ineffective. The first day or first few days after undergoing treatment may not create a decrease in fasting single breath methane concentrations. However, if after a few days (e.g., 3 or more days, 4 or more days, or 5 or more days), if the fasting single breath methane concentration compared to the initial breath methane concentration is stable (e.g., the same or substantially the same), it can be indicative that the IMO treatment is not effective.

In various embodiments, detecting a stable methane concentration in the fasting breath sample compared the previous breath methane concentration indicates that the treatment for IMO is effective. In situations wherein after several days of treatment (e.g., 3 or more days, 4 or more days, 5 or more days, or 7 or more days), the fasting single breath methane concentration is stable, it can indicate that the treatment is effective in that it brought down the methane concentration, and keeps the concentration at a stable level even though a further decrease is not seen.

In various embodiments, detecting an increase in the methane concentration in the fasting breath sample compared to the initial breath methane concentration, or compared to the previous breath methane concentration indicates that the treatment for IMO is ineffective. In certain instances, the methane concentration may initially fluctuate (e.g., after the first day it actually increases, but thereafter it decreases), and thus, if after the several days (e.g., 4 or more days, 5 or more days, 7 or more days), the methane concentration increases, it can indicate that the treatment is ineffective.

In various embodiments, detecting a methane concentration of less than 10 ppm is indicative of the treatment for IMO is effective.

In various embodiments, the method further comprises having the subject continue to treatment for IMO if the treatment is effective.

In various embodiments, the method further comprises having the subject stop the treatment for IMO if the treatment is ineffective.

In various embodiments, the method further comprises having the subject change the treatment for IMO if the treatment is ineffective. In various embodiments, the method further comprises having the subject change the treatment for IMO if there is recurrence of the disease.

In various embodiments, the method further comprises having the subject stop the treatment for IMO if the methane concentration is less than 10 ppm. As 10 ppm methane concentration can be used to diagnose IMO, concentrations less than 10 ppm can indicate that IMO has been treated and the treatment can be stopped. In various embodiments, a concentration of less than 10 ppm over a few days (e.g., 3, days, 4 days, 5 days, 6 days, or 7 days) would be indicative to stop treatment.

Various embodiments provide for a method of monitoring breath methane concentration in a subject, comprising: obtaining a single fasting breath sample from the subject, wherein the subject is undergoing treatment for intestinal methanogen overgrowth (IMO) or has undergone treatment for IMO; measuring the methane concentration in the single fasting breath sample; comparing the methane concentration to an initial breath methane concentration from the subject or a previous breath methane concentration from the subject, detecting a decrease in the methane concentration in the fasting breath sample compared to the initial breath methane concentration, or the previous breath methane concentration, or detecting a stable methane concentration in the fasting breath sample compared to the initial breath methane concentration, or the previous breath methane concentration, or detecting an increase in the methane concentration in the fasting breath sample compared to the initial breath methane concentration, or the previous breath methane concentration.

In various embodiments, the initial breath methane concentration is from the subject is from a lactulose breath test, from a glucose breath test, or from a fasting single methane measurement.

In various embodiments, the previous breath methane concentration from the subject is from a lactulose breath test, from a glucose breath test, or from a fasting single methane measurement.

In various embodiments, the method further comprises performing all the method steps two or more times. In various embodiments, the method can further comprise performing all the method steps 3, 4, 5, 6, 7, 8, 9, 10, 11, 12, 13, 14, 15, 16, 17, 18, 19, 20, or 21 or more times.

In various embodiments, the method further comprises comprising performing all the method steps two or more times for about 7-14 days. In various embodiments, the method further comprises comprising performing all the method steps two or more times for about 10 days. In various embodiments, the method further comprises comprising performing all the method steps two or more times for about 14-21 days.

In various embodiments, performing all the method steps comprising performing all the method steps once per day. In various embodiments, the method further comprises performing all the method steps twice per day. Performing the all the method steps multiple times per day can also be done (e.g., 3 times, 4 times, 5 times, etc.).

In various embodiment, obtaining the single fasting breath sample comprises having the subject collect their fasting breath samples at home every morning (before consuming any food) by blowing into a test tube, breath test collection bag or directly into a gas chromatographer. In various embodiment, obtaining the single fasting breath sample comprises having the subject collect their fasting breath samples after the subject has not ingested food for at least 4 hours by blowing into a test tube, breath test collection bag or directly into a gas chromatographer.

Various embodiments of the present invention provide for a method for treating intestinal methanogen overgrowth (IMO) in a subject, comprising: obtaining a fasting single breath methane concentration from a subject who is undergoing treatment for intestinal methanogen overgrowth (IMO) or has undergone treatment for IMO; comparing the fasting single breath methane concentration to an initial breath methane concentration from the subject or a previous breath methane concentration from the subject; and continuing the treatment for IMO if a decrease in the single fasting breath methane concentration compared to an initial breath methane concentration, or compared to a previous breath methane concentration is detected indicating that the treatment for IMO is effective, or stopping the treatment for IMO if a stable single fasting breath methane concentration compared to the initial breath methane concentration indicating that the treatment for IMO is ineffective, or continuing the treatment for IMO if a stable single fasting breath methane concentration compared to the previous breath methane concentration is detected indicating that the treatment for IMO is effective, or changing the treatment for IMO if an increase in the single fasting breath methane concentration compared to the initial breath methane concentration, or compared to the previous breath methane concentration is detected indicating that the treatment for IMO is ineffective, or stopping the treatment for IMO if a methane concentration of less than 10 ppm is detected.

Various embodiments of the present invention provide for a method for treating intestinal methanogen overgrowth (IMO) in a subject, comprising: obtaining a fasting single breath methane concentration from a subject who is undergoing treatment for intestinal methanogen overgrowth (IMO) or has undergone treatment for IMO; comparing the fasting single breath methane concentration to an initial breath methane concentration from the subject or a previous breath methane concentration from the subject; and continuing the treatment for IMO if a decrease in the single fasting breath methane concentration compared to an initial breath methane concentration, or compared to a previous breath methane concentration is detected indicating that the treatment for IMO is effective.

Various embodiments of the present invention provide for a method for treating intestinal methanogen overgrowth (IMO) in a subject, comprising: obtaining a fasting single breath methane concentration from a subject who is undergoing treatment for intestinal methanogen overgrowth (IMO) or has undergone treatment for IMO; comparing the fasting single breath methane concentration to an initial breath methane concentration from the subject or a previous breath methane concentration from the subject; and stopping the treatment for IMO if a stable single fasting breath methane concentration compared to the initial breath methane concentration is detected indicating that the treatment for IMO is ineffective.

Various embodiments of the present invention provide for a method for treating intestinal methanogen overgrowth (IMO) in a subject, comprising: obtaining a fasting single breath methane concentration from a subject who is undergoing treatment for intestinal methanogen overgrowth (IMO) or have undergone treatment for IMO; comparing the fasting single breath methane concentration to an initial breath methane concentration from the subject or a previous breath methane concentration from the subject; and continuing the treatment for IMO if a stable single fasting breath methane concentration compared to the previous breath methane concentration is detected indicating that the treatment for IMO is effective.

Various embodiments of the present invention provide for a method for treating intestinal methanogen overgrowth (IMO) in a subject, comprising: obtaining a fasting single breath methane concentration from a subject who is undergoing treatment for intestinal methanogen overgrowth (IMO) or has undergone treatment for IMO; comparing the fasting single breath methane concentration to an initial breath methane concentration from the subject or a previous breath methane concentration from the subject; and changing the treatment for IMO if an increase in the single fasting breath methane concentration compared to the initial breath methane concentration, or compared to the previous breath methane concentration is detected indicating that the treatment for IMO is ineffective.

Various embodiments of the present invention provide for a method for treating intestinal methanogen overgrowth (IMO) in a subject, comprising: obtaining a fasting single breath methane concentration from a subject who is undergoing treatment for intestinal methanogen overgrowth (IMO) or has undergone treatment for IMO; comparing the fasting single breath methane concentration to an initial breath methane concentration from the subject or a previous breath methane concentration from the subject; and stopping the treatment for IMO if a methane concentration of less than 10 ppm is detected.

Various embodiments of the present invention provide for a method of treating intestinal methanogen overgrowth (IMO) in a subject undergoing treatment for IMO, or has undergone treatment for IMO comprising: continuing the treatment for IMO if a decrease in a single fasting breath methane concentration compared to an initial breath methane concentration, or compared to a previous breath methane concentration is detected indicating that the treatment for IMO is effective, or stopping the treatment for IMO if a stable single fasting breath methane concentration compared to the initial breath methane concentration is detected indicating that the treatment for IMO is ineffective, or continuing the treatment for IMO if a stable single fasting breath methane concentration compared to the initial breath methane concentration, or compared the previous breath methane concentration is detected indicating that the treatment for IMO is effective, or changing the treatment for IMO if an increase in the single fasting breath methane concentration compared to the initial breath methane concentration, or compared to the previous breath methane concentration is detected indicating that the treatment for IMO is ineffective, or stopping the treatment for IMO if a methane concentration of less than 10 ppm is detected.

Various embodiments of the present invention provide for a method of treating intestinal methanogen overgrowth (IMO) in a subject undergoing treatment for IMO, or has undergone treatment for IMO comprising: continuing the treatment for IMO if a decrease in a single fasting breath methane concentration compared to an initial breath methane concentration, or compared to a previous breath methane concentration is detected indicating that the treatment for IMO is effective.

Various embodiments of the present invention provide for a method of treating intestinal methanogen overgrowth (IMO) in a subject undergoing treatment for IMO, or has undergone treatment for IMO comprising: stopping the treatment for IMO if a stable single fasting breath methane concentration compared to the initial breath methane concentration is detected indicating that the treatment for IMO is ineffective.

Various embodiments of the present invention provide for a method of treating intestinal methanogen overgrowth (IMO) in a subject undergoing treatment for IMO, or has undergone treatment for IMO comprising: continuing the treatment for IMO if a stable single fasting breath methane concentration compared to the initial breath methane concentration, or compared the previous breath methane concentration is detected indicating that the treatment for IMO is effective.

Various embodiments of the present invention provide for a method of treating intestinal methanogen overgrowth (IMO) in a subject undergoing treatment for IMO, or has undergone treatment for IMO comprising: changing the treatment for IMO if an increase in the single fasting breath methane concentration compared to the initial breath methane concentration, or compared to the previous breath methane concentration is detected indicating that the treatment for IMO is ineffective.

Various embodiments of the present invention provide for a method of treating intestinal methanogen overgrowth (IMO) in a subject undergoing treatment for IMO, or has undergone treatment for IMO comprising: stopping the treatment for IMO if a methane concentration of less than 10 ppm is detected.

In various embodiment, obtaining the single fasting breath sample comprises having the subject collect their fasting breath samples at home every morning (before ingesting food) by blowing into a test tube, breath test collection bag or directly into a gas chromatographer. In various embodiment, obtaining the single fasting breath sample comprises having the subject collect their fasting breath samples after the subject has not ingested food for at least 4 hours by blowing into a test tube, breath test collection bag or directly into a gas chromatographer.

Various embodiments of the present invention provide for a method of confirming eradication or lack of eradication of intestinal methanogen overgrowth (IMO), comprising: obtaining a single fasting breath sample from the subject, measuring the methane concentration in the single fasting breath sample; and detecting a methane concentration in the fasting breath sample of less than 10 ppm. In various embodiments, the subject is undergoing treatment for IMO. In various embodiments, the subject has undergone treatment for IMO.

In various embodiments, detecting a methane concentration in the fasting breath sample of less than 10 ppm comprises detecting a methane concentration in the fasting breath sample of less than 9 ppm. In various embodiments, detecting a methane concentration in the fasting breath sample of less than 10 ppm comprises detecting a methane concentration in the fasting breath sample of less than 8 ppm. In various embodiments, detecting a methane concentration in the fasting breath sample of less than 10 ppm comprises detecting a methane concentration in the fasting breath sample of less than 7 ppm. In various embodiments, detecting a methane concentration in the fasting breath sample of less than 10 ppm comprises detecting a methane concentration in the fasting breath sample of less than 6 ppm. In various embodiments, detecting a methane concentration in the fasting breath sample of less than 10 ppm comprises detecting a methane concentration in the fasting breath sample of less than 5 ppm.

In various embodiment, obtaining the single fasting breath sample comprises having the subject collect their fasting breath samples at home every morning by blowing into a test tube, breath test collection bag or directly into a gas chromatographer. In various embodiment, obtaining the single fasting breath sample comprises having the subject collect their fasting breath samples after the subject has not ingested food for at least 4 hours by blowing into a test tube, breath test collection bag or directly into a gas chromatographer.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Patient Population and Study Design

To assess test characteristics of SMM, two separate databases were analyzed: 1) Consecutive lactulose breath tests performed from November 2005 to October 2013 at Cedars-Sinai, Los Angeles, CA; 2) Glucose breath tests performed from January 2007 to December 2015 at the Medical College of Georgia, Augusta, GA. Repeat studies on the same subject were excluded. The sensitivity, specificity, positive predictive value (PPV), and negative predictive value (NPV) of diagnosing IMO based on the various SMM cutoffs (3-10 ppm) as compared to all measurements during the 2-hour test were calculated. The margin of error for the gas chromatography in measuring CH₄ was ±2 ppm; thus test characteristics for 1 and 2 ppm were not calculated. The test characteristics of the lactulose breath test and glucose breath tests were compared.

The glucose breath test database included baseline symptoms profiles for each subject. Subjects were given a previously validated questionnaire assessing 10 common gastrointestinal symptoms Subjects reported the severity of their symptoms by grading the frequency, intensity, and duration on a scale of 0-3. The scores were added resulting in a maximum score of 9 and a minimum score of 0. (18-20).

To evaluate the accuracy and temporal stability of SMM, and to assess its correlation with stool M. smithii loads, we analyzed data from a randomized double-blind placebo-controlled trial: Efficacy and Safety of Single, Daily Oral Doses of SYN-010 Compared to Placebo in Adult Patients with IBS-C (EASE-DO, NCT03763175).

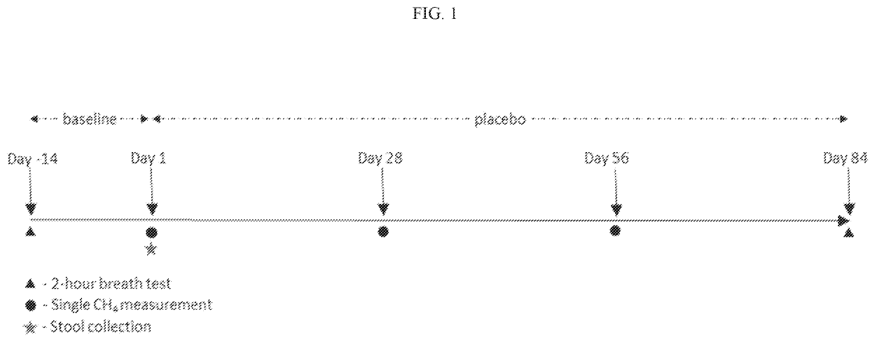

Trial details: Efficacy and Safety of Single, Daily Oral Doses of SYN-010 Compared to Placebo in Adult Patients with IBS-C (EASE-DO, NCT03763175) was a 14-week single-center randomized double-blind placebo-controlled trial evaluating the effects of lovastatin lactone on subjects with IBS-C and IMO. Subjects were excluded if they were on statins, fibrates, niacin, narcotics, laxatives, tegaserod, lubiprostone, linaclotide, metoclopramide, prucalopride, domperidone, plecanatide, CandiBactin, Atrantil, Allimax/Allimed, antibiotics within 2 months and opioids within 3 months prior to the study. If subjects were on probiotics or fiber supplements, they were asked to not change their dose. The trial was terminated early for not meeting the primary endpoint during an interim futility analysis. All subjects enrolled in this study completed a 2-hour breath test at their initial visit and their weekly complete spontaneous bowel movement (CSBM) and spontaneous bowel movement (SBM) were recorded. After recording their baseline CSBM and SBM for 2 weeks, subjects returned to the lab and their SMM and stool samples were collected (FIG. 1). Subjects were randomized to receive the study drug or placebo in a 2:1 fashion and continued recording their weekly CSBM, SBM. Subsequently, a SMM was collected on day 28 and 56 and a full 2-hour lactulose breath test was performed at the completion on day 84.

SMM measured on day 1 was compared to a 2-hour breath test performed on day −14 of the trial (i.e. prior to any interventions). Specifically, we calculated the sensitivity of SMM on day 1 for diagnosing IMO as compared to that of a 2-hour breath test on day −14.

Next, to investigate the stability of SMM over an extended period, we analyzed SMM collected over 14 weeks in subjects who received placebo. SMM were assessed on days 1, 28 and 56, and compared to baseline breath samples (before lactulose administration) from 2-hour breath tests performed on days −14 and 84. In addition, stool collected on day 1 was analyzed for M. smithii load and compared to SMM levels from the same day.

For the final aim of this study, adult subjects with an IMO diagnosis based on a 2-hour breath test and undergoing antibiotic therapy were included. Upon initiation of antibiotics, SMM was collected every morning for 10 days and were analyzed.

All studies were approved by the corresponding institutional review boards.

### Breath Testing

For the full 2-hour breath tests, subjects were asked to consume a low-fermentable diet starting 24-hours prior to the breath test, then fast for the last 12 hours. On arriving to the lab, a breath sample was collected in a single-patient breath collection. Then, subjects were asked to ingest 10 g of lactulose or 75 g of glucose and breath samples were collected every 15-minutes for 120 minutes. CH₄. H₂ and CO₂ concentrations were measured using gas chromatography (Gemelli Biotech, Los Angeles, CA, for subjects in EASE-DO trial and Quintron, Milwaukee, WI, for all other subjects). CH₄ and H₂ concentrations were adjusted to alveolar CO₂ concentration of 5.5%.

Patients undergoing daily breath sampling collected their fasting breath samples at home every morning by blowing into a test tube (Extainer, Labco, Ceredigion, UK).

### Stool Sample Collection and DNA Extraction

Patients collected their stool at home within 48 hours of their appointment using a Fisherbrand™ Commode Specimen Collection System (Thermo Fisher Scientific, MA, USA). After samples were received at the lab, they were immediately transferred to OMNIgene•GUT tubes (DNA Genotek, Ottawa, ON, Canada). DNA extraction was carried out using the MagAttract PowerSoil DNA KF Kit (Qiagen, cat. No. 27000-4-KF) with some modifications. DNA quality and concentration were determined using a NanoDrop One spectrophotometer (Thermo Fisher Scientific, MA, USA). DNA was also extracted from an M. smithii stock culture following the same steps.

### Quantitative POR Assay

Stool DNA samples were diluted to 25 ng/μL with EB buffer (Qiagen). M. smithii DNA loads were determined by qPCR using the gene encoding the beta subunit of RNA polymerase (rpoB) as the target gene. The specific primers and probe used for M. smithii were described by Dridi et al. Primers and probe were optimized by Applied Biosystems (Custom Taqman Gene Expression Assays). qPCR was performed on a QuantStudio 6 Flex System (Thermo Fisher Scientific, Waltham, Massachusetts, USA) DNA extracted from an M. smithii stock culture was measured at 11.25 ng/μL. This was further diluted to 1.13×10⁻⁷ ng/μL after a series of tenfold dilutions and used to establish a standard curve. The limit of detection was found to be at 1.13×10⁻⁴ ng/μL. M. smithii load was expressed as ng of M. smithii per mg of stool.

### Statistical Analysis

Continuous variables were summarized as mean±standard deviation and categorical variables were summarized as count (%). Sensitivity, specificity, positive predictive value (PPV), negative predictive value (NPV), positive likelihood ratio, and negative likelihood ratio of every cutoff value from 3 to 10 ppm for SMM was compared to the gold-standard CH₄≥10 ppm on a 2-hour breath test, using a 2×2 contingency table. An a priori difference of <3% was assumed to be clinically similar when comparing the test characteristics of SMM. Symptomology was analyzed using Wilcoxon-Rank Sum Test. Change in SMM, complete spontaneous bowel movement (CSBM), and spontaneous bowel movement (SBM) over time was analyzed using the repeated ANOVA test. To see if there were any interaction between the change in SMM and bowel movement (BM), we performed a two-step analysis where we first modeled the respective variables over time using linear regression. Then the slopes of these lines were compared using Pearson's correlation. For statistical purposes when comparing SMM to M. smithii, any undetectable load of M. smithii on qPCR was replaced by the lowest detectable M. smithii level divided by 2. M. smithii loads were normalized using natural log transformation prior to analysis and compared to SMM using Pearson's correlation. All analyses were performed using SAS 9.4 and a two-tailed alpha of <0.05 was used to define significance.

### Results

### SMM Accurately Diagnoses IMO on the 2-Hour Lactulose Breath Test.

Of 14,847 lactulose breath tests, 2,664 repeat breath tests were excluded, leaving 12,183 unique subjects. 1891 (15.5%) were deemed to have IMO based on the 2-hour test. To diagnose IMO, various cutoffs for SMM (3-10 ppm) were assessed where the sensitivity ranged from 86.4% to 98.8% and specificity ranged from 99.3% to 100% (Table 1).

TABLE 1
Test performance for SMM compared to the 2-hour lactulose breath test

| | | | | | | | EASE-DO |
|---|---|---|---|---|---|---|---|
| SMM | Sensitivity | Specificity | PPV | NPV | | | Sensitivity |
| (ppm) | (95% CI) | (95% CI) | (95% CI) | (95% CI) | +LR | −LR | (95% CI) |
| ≥10 | 86.4* | 100 | 100 | 97.6 | N/A | 0.14 | 84.8* |
| | (84.8-87.9) | | (99.8-100) | (97.3-97.8) | | | (75.6-93.9) |
| ≥9 | 88.8 | 100 | 99.9 | 98 | 4569 | 0.11 | 84.8 |
| | (87.3-90.2) | (99.9-100) | (99.6-100) | (97.7-98.2) | | | (75.6-93.9) |
| ≥8 | 90.7 | 99.9 | 99.7 | 98.3 | 1557 | 0.09 | 84.8 |
| | (89.3-92) | (99.9-100) | (99.2-99.9) | (98.1-98.6) | | | (75.6-93.9) |
| >7 | 93.0 | 99.9 | 99.3 | 98.7 | 736 | 0.07 | 84.8 |
| | (91.8-94.1) | (99.8-99.9) | (98.7-99.6) | (98.5-98.9) | | | (75.6-93.9) |
| ≥6 | 94.6 | 99.7 | 99.1 | 99 | 572 | 0.05 | 86.4 |
| | (93.4-95.5) | (99.6-99.8) | (98.5-99.5) | (98.8-99.2) | | | (77.7-95.2) |
| ≥5 | 96.1 | 99.7 | 98.5 | 99.3 | 353 | 0.04 | 86.4 |
| | (95.1-96.9) | (99.6-99.8) | (97.8-99.0) | (99.1-99.4) | | | (77.7-95.2) |
| ≥4 | 97.3 | 99.6 | 97.7 | 99.5 | 227 | 0.03 | 89.8 |
| | (96.4-97.9) | (99.4-99.7) | (96.9-98.3) | (99.3-99.6) | | | (82.1-97.5) |
| ≥3 | 98.8 | 99.3 | 96 | 99.8 | 132 | 0.01 | 91.5 |
| | (98.2-99.3) | (99.1-99.4) | (95.1-96.9) | (99.7-99.9) | | | (84.4-98.6) |

Test characteristics of SMM (initial fasting CH₄ prior to lactulose) diagnosing IMO based on the 2-hour breath test (n=12,183). EASE-DO sensitivity represents SMM taken on Day 1 compared to IMO diagnosed on Day −14 on a 2-hour breath test. *Clinically similar between the lactulose breath test and the EASE-DO cohort based on a difference of <3% set as a priori. SMM=single CH₄ measurements, CH₄=methane, IMO-intestinal methanogen overgrowth.

### SMM Accurately Diagnoses IMO on the 2-Hour Glucose Breath Test.

To assess whether the SMM is accurate for diagnosing IMO on the glucose breath test, a database of 733 subjects was analyzed. Of 733 subjects, 147 (20.05%) had IMO. The sensitivity rates of various SMM cutoffs (3-10 ppm) ranged from 86.4% to 98.8% and specificity ranged from 81.0% to 100% (Table 2). When SMM test characteristics during glucose breath testing were compared to the lactulose breath tests, sensitivity and specificity were clinically similar for the cutoffs 3-10 ppm and 8-10 ppm, respectively.

TABLE 2
Test performance for SMM compared to the 2-hour glucose breath test

| SMM | Sensitivity | Specificity | PPV | NPV | | |
|---|---|---|---|---|---|---|
| (ppm) | (95% CI) | (95% CI) | (95% CI) | (95% CI) | +LR | −LR |
| ≥10 | 86.4*† | 100† | 100 | 97.0 | N/A | 0.14 |
| | (80.9-91.9) | | (100-100) | (95.7-98.3) | | |
| ≥9 | 88.4† | 99.4† | 97.0 | 97.4 | 147.3 | 0.12 |
| | (83.3-93.6) | (98.8-100) | (94.1-99.9) | (96.2-98.6) | | |
| ≥8 | 92.5† | 98.5† | 93.2 | 98.3 | 61.7 | 0.08 |
| | (88.3-96.8) | (97.5-99.4) | (89.1-97.3) | (97.3-99.3) | | |
| ≥7 | 93.9† | 96.6 | 86.3 | 98.6 | 27.6 | 0.06 |
| | (90.0-97.8) | (95.2-98.0) | (80.9-91.6) | (97.7-99.5) | | |
| ≥6 | 94.6† | 94.2 | 79.0 | 98.7 | 16.3 | 0.06 |
| | (90.9-98.2) | (92.5-96.1) | (73.0-85.0) | (97.8-99.6) | | |
| ≥5 | 95.2† | 91.0 | 70.7 | 98.8 | 10.6 | 0.05 |
| | (91.8-98.7) | (88.8-93.2) | (64.4-77.1) | (98.0-99.7) | | |
| ≥4 | 97.3† | 87.8 | 64.4 | 99.3 | 8.0 | 0.03 |
| | (94.7-99.9) | (85.2-90.3) | (58.1-70.7) | (98.6-100) | | |
| ≥3 | 99.3 † | 81.0 | 54.3 | 99.8 | 5.2 | 0.009 |
| | (98.0-100) | (77.9-84.0) | (48.3-60.2) | (99.4-100) | | |

Test characteristics of SMM (initial fasting CH₄ prior to glucose) diagnosing IMO based on the 2-hour breath test (n=733). *Clinically similar between the glucose breath test cohort and the EASE-DO cohort based on a difference of 3% set as a priori. †Clinically similar between the glucose breath test cohort and the lactulose breath test cohort based on a difference of 3% set as a priori. SMM=single CH₄ measurements, CH₄=methane, IMO=intestinal methanogen overgrowth.

SMM Performed 2 Weeks after a 2-Hour Lactulose Breath Test Accurately Diagnoses IMO.

To assess the accuracy of SMM to diagnose IMO based on a 2-hour breath test performed on a separate day, SMM test characteristics from subjects in the EASE-DO trial on day 1 were compared to the 2-hour breath test on day −14 (n=59). Subjects received no treatment during the screening phase of the trial between day −14 and day 1. The sensitivity rates of SMM cutoffs from 3-10 ppm were 84.8%-91.5% (Table 1). The sensitivity of SMM ≥10 ppm for diagnosing IMO in this cohort was similar to the sensitivities from both the lactulose and glucose breath test databases using the same cutoff. Specificity could not be assessed for various cut-offs given that all subjects in the EASE-DO cohort had IMO.

SMM ≥10 ppm is Associated with Constipation, Gas, and Less Diarrhea

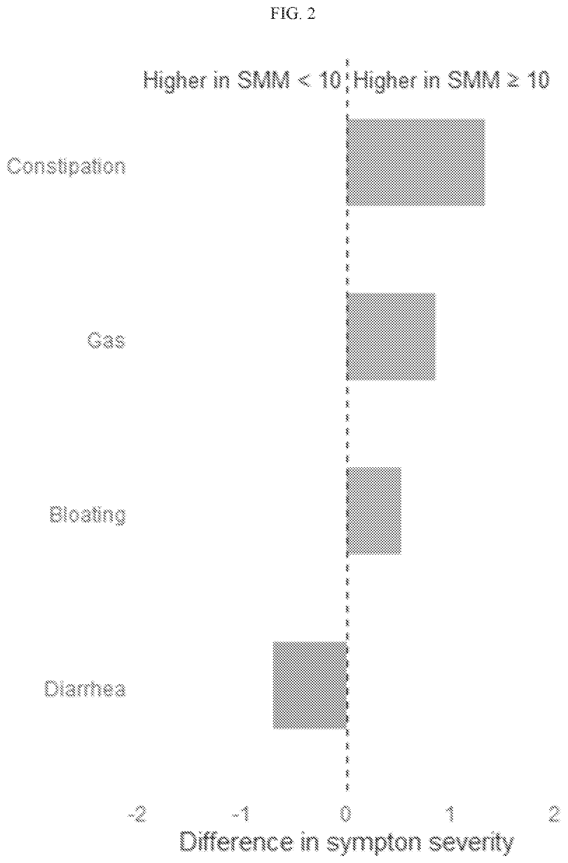

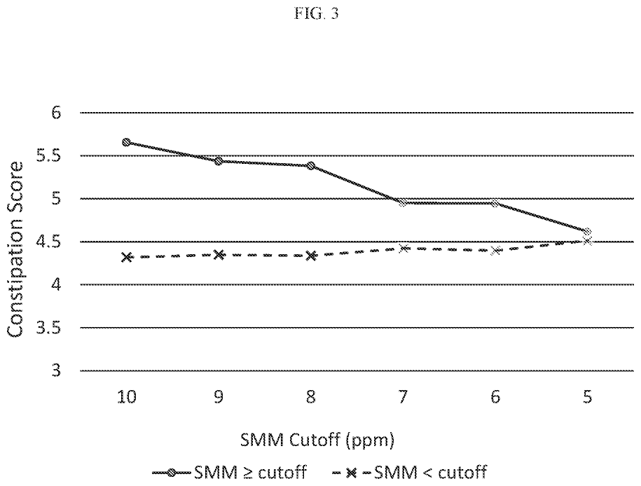

In the glucose breath test database, 732 subjects reported on all symptoms except constipation, for which data were available for 338 subjects. Subjects with SMM ≥10 ppm reported higher scores for constipation (5.65±3.47 vs 4.32±3.62, p=0.008) and gas (6.27±2.77 vs 5.41±2.98, p=0.003), while reporting a lower score for diarrhea (3.68±3.49 vs 4.38±3.46, p=0.04) (FIG. 2). There was a trend towards higher bloating severity in the SMM ≥10 ppm group which did not reach statistical significance (6.24±3.29 vs 5.71±3.36, p=0.059). Other symptoms were statistically similar (Table 3). Using the cutoffs 10, 9, and 8 ppm, there were statistically significant differences in the reported constipation scores (FIG. 3). At SMM of 7 ppm, the difference in constipation severity between the two groups was not significant (4.95±3.66 vs 4.42±3.62, p=0.23). Similarly, at SMM <7 ppm the difference in constipation severity was not significant. At 9 ppm there was no significant difference in diarrhea severity for those with SMM ≥9 ppm vs SMM <9 ppm (3.73±3.50 vs 4.39±3.46, p=0.06). The SMM cutoff of 10 ppm provided the largest difference in symptomology.

TABLE 3
Symptomology of subjects using the cutoff SMM ≥ 10 ppm

| | SMM ≥ 10 | SMM < 10 | p-value |
|---|---|---|---|
| Constipation | 5.66 ± 3.46 | 4.31 ± 3.62 | 0.008 |
| Bloating | 6.24 ± 3.29 | 5.71 ± 3.36 | 0.059 |
| Abdominal Pain | 5.54 ± 3.23 | 5.75 ± 3.09 | 0.46 |
| Diarrhea | 3.68 ± 3.48 | 4.39 ± 3.45 | 0.04 |
| Vomiting | 1.51 ± 2.77 | 1.61 ± 2.70 | 0.48 |
| Gas | 6.27 ± 2.76 | 5.41 ± 2.98 | 0.003 |
| Belching | 3.96 ± 3.15 | 3.79 ± 3.14 | 0.53 |
| Nausea | 3.91 ± 3.53 | 4.09 ± 3.41 | 0.58 |
| Indigestion | 4.7 ± 3.53 | 4.32 ± 3.42 | 0.23 |
| Fullness | 5.71 ± 3.17 | 5.32 ± 3.38 | 0.33 |

Composite score of each symptom calculated as the combination of intensity, duration, and frequency graded on a scale of 0-3, with higher numbers representing higher severity, increased duration and increased frequency respectively. Subjects with SMM ≥10 reported a higher score on constipation, and gas and a lower diarrhea score.
Subjects with IMO Who do not Receive Active Treatment have Stable SMM Over Time.

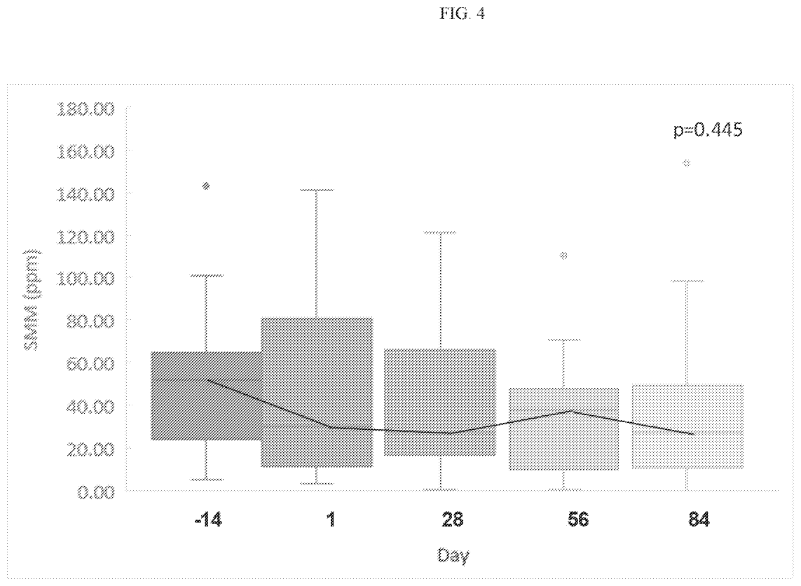

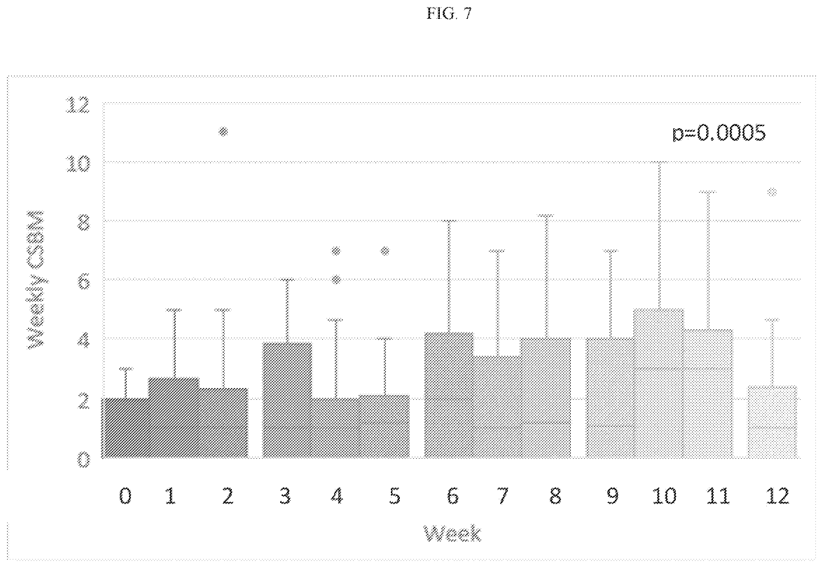

To measure the stability of SMM over time, we analyzed SMM over time in the placebo arm of the Ease-Do cohort (n=20). The cohort had a mean age of 48.10±9.26 years and 14 subjects (70%) were female. Baseline SMM was 50.07±33.03 ppm. There was no significant decrease in SMM over time (p=0.45, FIG. 4). During the study, no change in SBM was seen in the placebo group (p=0.45), while CSBM changed over time (p=0.0005, FIG. 7). There was no correlation between CSBM and SMM (R=0.23, p=0.33).

SMM Correlates with Stool M. smithii DNA Load

SMM and stool samples were available from 28 subjects on day 1 from the EASE-DO trial. Average SMM was 49.73±37.45 ppm and the average M. smithii DNA concentration was 0.80±1.14 ng per mg of stool. There was a significant positive correlation between SMM and stool M. smithii DNA load (R=0.65, p=0.0002, FIG. 5). This may be due to subjective component of “completeness” when assessing CSBM which may be more prone to a placebo response.

### Daily SMM Decreases During Antibiotic Therapy.

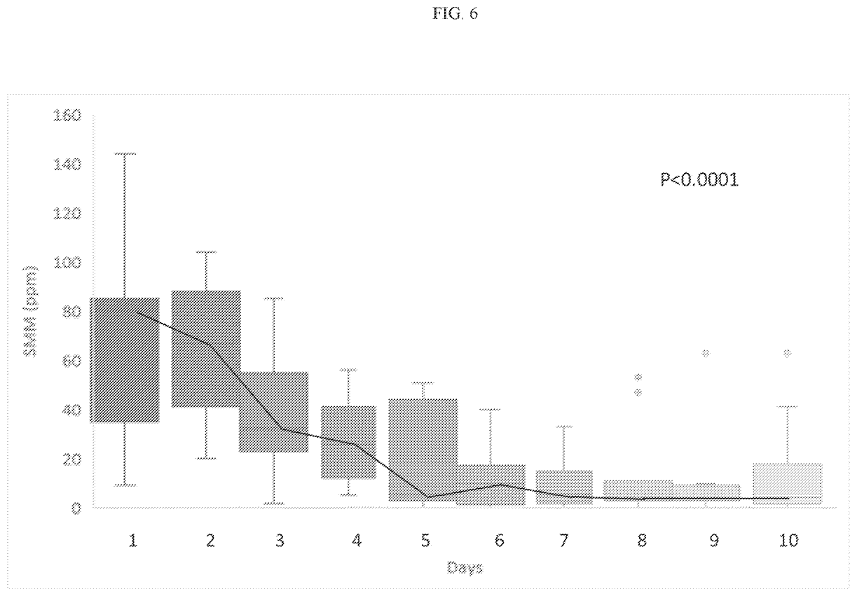

Lastly, 11 subjects with IMO (45.5±14.9 years and 7 (63.6%) female) were recruited to measure the change in SMM after antibiotic therapy. Nine (81.8%) subjects received rifaximin and neomycin while 1 (9.1%) received neomycin alone and another received neomycin and metronidazole. Mean baseline CH₄ was 69.9±35.2 ppm which showed significant decrease on daily SMM while receiving antibiotics (P<0.0001) (FIG. 6). By day 3, SMM was significantly different compared to baseline (p<0001) (Table 4).

TABLE 4
Coefficient estimate on each day after initiation of antibiotics

| Variable | Estimate | P value |
|---|---|---|
| Day 1 | reference | reference |
| Day 2 | −7.182 | 0.3557 |
| Day 3 | −31.636 | <.0001 |
| Day 4 | −44.909 | <.0001 |
| Day 5 | −52.273 | <.0001 |
| Day 6 | −57.909 | <.0001 |
| Day 7 | −59.636 | <.0001 |
| Day 8 | −57.364 | <.0001 |
| Day 9 | −60.364 | <.0001 |
| Day 10 | −57.000 | <.0001 |

By day 3 there was a significant drop in SMM levels for those on antibiotics

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A method of assessing treatment response in a subject, comprising:
obtaining a fasting single breath sample from the subject, wherein the subject is undergoing treatment for intestinal methanogen overgrowth (IMO) or has undergone treatment for IMO;
measuring the methane concentration in the fasting single breath sample;
comparing the methane concentration to an initial breath methane concentration from the subject or a previous breath methane concentration from the subject,
detecting a decrease in the methane concentration in the fasting breath sample compared to the initial breath methane concentration, or compared to the previous breath methane concentration, or
detecting a stable methane concentration in the fasting breath sample compared to the initial breath methane concentration, or compared the previous breath methane concentration, or
detecting an increase in the methane concentration in the fasting breath sample compared to the initial breath methane concentration, or compared to the previous breath methane concentration, or
detecting a methane concentration of less than 10 ppm.

2. The method of claim 1, wherein the initial breath methane concentration from the subject is from a lactulose breath test, from a glucose breath test, or from a single fasting single methane measurement, or wherein the previous breath methane concentration from the subject is from a lactulose breath test, from a glucose breath test, or from single a fasting single methane measurement;

3. A method of claim 1, further comprising performing all the method steps two or more times.

4. A method of claim 1, further comprising performing all the method steps two or more times for about 7-14 days.

5. A method of claim 1, further comprising performing all the method steps two or more times for about 10 days.

6. A method of claim 1, wherein all the method steps are performed once per day.

7. A method of claim 1, wherein
detecting a decrease in the methane concentration in the fasting breath sample compared to the initial breath methane concentration, or compared to the previous breath methane concentration indicates that the treatment for IMO is effective, or
detecting a stable methane concentration in the fasting breath sample compared to the initial breath methane concentration indicates that the treatment for IMO is ineffective, or
detecting a stable methane concentration in the fasting breath sample compared the previous breath methane concentration indicates that the treatment for IMO is effective, or
detecting an increase in the methane concentration in the fasting breath sample compared to the initial breath methane concentration, or compared to the previous breath methane concentration indicates that the treatment for IMO is ineffective, or
detecting a methane concentration of less than 10 ppm is indicative of the treatment for IMO is effective.

8. The method of claim 7, further comprising having the subject continue to treatment for IMO if the treatment is effective.

9. The method of claim 7, further comprising having the subject stopping the treatment for IMO if the treatment is ineffective.

10. The method of claim 7, further comprising having the subject change the treatment for IMO if the treatment is ineffective or there is recurrence of IMO.

11. The method of claim 7, further comprising having the subject stop the treatment for IMO if the methane concentration is less than 10 ppm.

12. A method of monitoring breath methane concentration in a subject, comprising:
obtaining a single fasting breath sample from the subject, wherein the subject is undergoing treatment for intestinal methanogen overgrowth (IMO) or has undergone treatment for IMO;
measuring the methane concentration in the single fasting breath sample;
comparing the methane concentration to an initial breath methane concentration from the subject or a previous breath methane concentration from the subject,
detecting a decrease in the methane concentration in the fasting breath sample compared to the initial breath methane concentration, or the previous breath methane concentration, or
detecting a stable methane concentration in the fasting breath sample compared to the initial breath methane concentration, or the previous breath methane concentration, or
detecting an increase in the methane concentration in the fasting breath sample compared to the initial breath methane concentration, or the previous breath methane concentration.

13. The method of claim 12, wherein the initial breath methane concentration from the subject is from a lactulose breath test, from a glucose breath test, or from a fasting single methane measurement, or wherein the previous breath methane concentration from the subject is from a lactulose breath test, from a glucose breath test, or from a fasting single methane measurement.

14. A method of claim 12, further comprising performing all the method steps two or more times.

15. A method of claim 12, further comprising performing all the method steps two or more times for about 7-14 days.

16. A method of claim 12, further comprising performing all the method steps two or more times for about 10 days.

17. A method of claim 12, wherein performing all the method steps comprising performing all the method steps once per day.

18. A method for treating intestinal methanogen overgrowth (IMO) in a subject, comprising:
obtaining a fasting single breath methane concentration from a subject who is undergoing treatment for intestinal methanogen overgrowth (IMO);
comparing the fasting single breath methane concentration to an initial breath methane concentration from the subject or a previous breath methane concentration from the subject; and
continuing the treatment for IMO if a decrease in the single fasting breath methane concentration compared to an initial breath methane concentration, or compared to a previous breath methane concentration is detected indicating that the treatment for IMO is effective, or
stopping the treatment for IMO if a stable single fasting breath methane concentration compared to the initial breath methane concentration is detected indicating that the treatment for IMO is ineffective, or
continuing the treatment for IMO if a stable single fasting breath methane concentration compared to the previous breath methane concentration is detected indicating that the treatment for IMO is effective, or
changing the treatment for IMO if an increase in the single fasting breath methane concentration compared to the initial breath methane concentration, or compared to the previous breath methane concentration is detected indicating that the treatment for IMO is ineffective, or
stopping the treatment for IMO if a methane concentration of less than 10 ppm is detected.

19. A method of treating intestinal methanogen overgrowth (IMO) in a subject undergoing treatment for IMO, comprising:
continuing the treatment for IMO if a decrease in a single fasting breath methane concentration compared to an initial breath methane concentration, or compared to a previous breath methane concentration is detected indicating that the treatment for IMO is effective, or
stopping the treatment for IMO if a stable single fasting breath methane concentration compared to the initial breath methane concentration is detected indicating that the treatment for IMO is ineffective, or
continuing the treatment for IMO if a stable single fasting breath methane concentration compared to the previous breath methane concentration is detected indicating that the treatment for IMO is effective, or
changing the treatment for IMO if an increase in the single fasting breath methane concentration compared to the initial breath methane concentration, or compared to the previous breath methane concentration is detected indicating that the treatment for IMO is ineffective, or
stopping the treatment for IMO if a methane concentration of less than 10 ppm is detected.

20. A method of confirming eradication or lack of eradication of intestinal methanogen overgrowth (IMO), comprising:
obtaining a single fasting breath sample from the subject, measuring the methane concentration in the single fasting breath sample; and
detecting a methane concentration in the fasting breath sample of less than 10 ppm to confirm eradication of IMO, or
detecting a methane concentration in the fasting breath sample of ≥10 ppm to confirm the lack of eradication of IMO.

21. The method of claim 20, wherein the subject is undergoing treatment for IMO.

22. The method of claim 20, wherein the subject has undergone treatment for IMO.
